(19) United States
(12) Patent Application Publication
(10) Pub. No.: US 2025/0332312 A1
(43) Pub. Date: Oct. 30, 2025

(54) MULTI-PORE AROMA DIFFUSER DEVICE

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 18/650,050

(22) Filed: Apr. 29, 2024

(51) Int. Cl. A61L 9/03; A61L 9/04

(52) U.S. Cl. CPC A61L 9/03; A61L 9/042; A61L 2209/135; A61L 2209/21

(57) ABSTRACT

A multi-pore aroma diffuser device includes an accommodation base and a porous diffuser to be detachably assembled on the accommodation base to replace existing diffuser stones. Through the porous material structure and a liquid storage bottle inside the device, fragrance liquid is continuously provided for the porous diffuser to continuously emit fragrance, and there is no need to rely on electricity or energy-consuming heating to produce fragrance. It has a long service life and slowly and controlled release of fragrance. The porous diffuser is detachably assembled on the accommodation base. The detachable design allows for easy replacement of the liquid storage bottle. It also greatly simplifies the weight and complexity of the overall structure, making it easy to use and conducive to releasing the effectiveness of the fragrance.

### BACKGROUND OF THE INVENTION

### 1. Field of the Invention

The present invention relates to the field of aroma release devices, in particular to a multi-pore aroma diffuser device, which has an aroma diffusion structure that continuously and appropriately releases liquid scent.

### 2. Description of the Related Art

For various liquid fragrances or perfumes such as essential oils, the more environmentally friendly way is to produce fragrance through natural evaporation. Fragrance liquids such as essential oils are dropped on porous aroma diffusion stones to evaporate in the space to produce various effects such as aroma atmosphere, aromatherapy or deodorization on the environment. The problem with natural evaporation is that when the user drops essential oil on the aroma diffusion stone, the natural evaporation rate of the fragrance is too slow or too fast to control the proper evaporation of the essential oil. The fragrance is not strong enough and the fragrance diffusion range is small. Furthermore, users may need to replenish essential oil drops on these aroma diffusion stones again and again. Essential oil diffusion is not sustainable and is inconvenient to use. These are disclosed in, for example, USD708,312 S, U.S. Pat. No. 8,265,465 B2, U.S. Pat. No. 8,787,739 B2, US20140366778 A1. Other evaporation carriers such as wheat straw or foam evaporate too quickly, making it difficult to preserve essential oils and easily wasted. Or the ceramic rods used by some diffusers to absorb essential oils break easily, interrupting the diffuser function. However, electronic or candle-heated diffusers consume less energy and are less environmentally friendly and have safety concerns.

### SUMMARY OF THE INVENTION

It is one object of the present invention to provide a multi-pore aroma diffuser device, which can continuously provide fragrance liquid through the structure of porous materials and a liquid storage bottle, so as to continuously emit fragrance from the multi-pore aroma diffuser device.

It is another object of the present invention to provide a multi-pore aroma diffuser device, which uses a porous structure to enable the aroma diffuser device to be lightweight and have excellent aroma liquid carrying and volatilization effects and enable the fragrance diffusion rate to be slow or controllable.

It is another object of the present invention to provide a multi-pore aroma diffuser device, in which the liquid storage bottle and structure of the aroma diffuser device are detachable to facilitate the use and replacement of the liquid storage bottle.

To achieve these and other objects of the present invention, the multi-pore aroma diffuser device comprises an accommodation base, a porous diffuser, and a liquid-conducting member. The accommodation base comprises a connecting surface formed on the upper side thereof, and a first opening opened in the middle of the connecting surface. The first opening extends inward to form a first accommodation space. The porous diffuser is used to absorb and evaporate functional liquids, such as fragrance liquids. The porous diffuser comprises a first top surface, a first bottom surface relative to the first top surface, a second opening formed in the middle of the first bottom surface, a second accommodation space hollowed inward from the second opening, a conductive portion formed in the top side within the second accommodation space, and a mounting portion located on the conductive portion. The capillary action of the porous structure of the conductive portion can connect the inner wall of the second accommodation space to the entire porous diffuser. The porous diffuser utilizes the first bottom surface to detachably combine with the connecting surface of the accommodation base. The liquid-conducting member is a porous material. The liquid-conducting member has one side thereof combined with the mounting portion, and an opposing free end side thereof passed through the first opening and the second opening and protruded into the accommodating space.

Preferably, the porous diffuser is, for example, a porous ceramic structure formed by firing clay between 700° C. and 1100° C., which has excellent adsorption and release effects of liquids, can reduce weight. At the same time, the overall structure can be greatly simplified. It is lightweight and has excellent fragrance liquid carrying and evaporation effects. The diffusion of fragrance can be slow or controlled at a release rate.

In summary, the multi-pore aroma diffuser device of the present invention can replace the existing aroma diffuser stones. By continuously providing fragrance liquid through the porous material structure and the liquid storage bottle, the fragrance is continuously emitted from the multi-pore aroma diffuser device to improve the problem of the diffuser stone. There is no need to rely on power-consuming or energy-consuming heating incense machines to produce fragrance. It has a long service life, is energy-saving and environmentally friendly. In some embodiments, porous structures, such as porous ceramic diffusers, are used to simultaneously absorb various liquids, such as functional liquids, such as essential oils or perfumes or other liquid fragrances, for adsorption and diffusion effects. The diffusion of fragrance can be slow or controlled at a release rate. The liquid storage bottle and structure of the device are detachable, making it easy to use and replace the liquid storage bottle, while also greatly simplifying the weight and complexity of the overall structure.

### BRIEF DESCRIPTION OF THE DRAWINGS

FIG. 1 is a perspective appearance view of a multi-pore aroma diffuser device according to the first preferred embodiment of the present invention.

FIG. 2 is an exploded view of a multi-pore aroma diffuser device according to the second preferred embodiment of the present invention.

FIG. 3 is an exploded elevation view of the multi-pore aroma diffuser device according to the second preferred embodiment of the present invention.

FIG. 4 is a cross-sectional view of the multi-pore aroma diffuser device according to the second preferred embodiment of the present invention.

FIG. 5 is a cross-sectional view of a multi-pore aroma diffuser device with a built-in liquid storage bottle according to the third preferred embodiment of the present invention.

### DETAILED DESCRIPTION OF THE PREFERRED EMBODIMENT

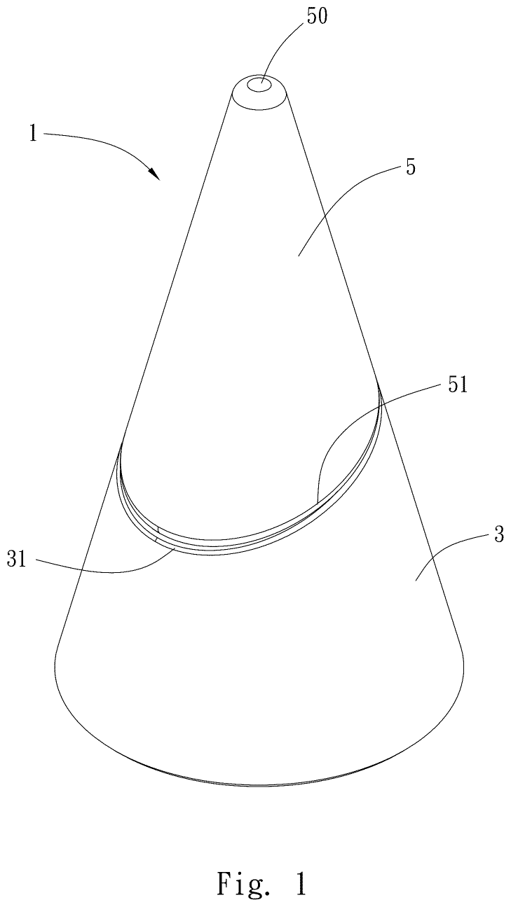

Referring to FIGS. 1-4, a multi-pore aroma diffuser device 1 according to an embodiment of the present invention comprises an accommodation base 3, a porous diffuser 5, and a liquid-conducting member 7. The accommodation base 3 comprises a connecting surface 31 formed on the upper side, a first opening 33 opened in the middle of the connecting surface 31. and a first accommodation space 35 formed therein in communication with the first opening 33.

The porous diffuser 5 is used to absorb and evaporate functional liquids. The porous diffuser 5 comprises a first top surface 50, a first bottom surface 51 opposite to the first top surface 50, a second opening 53 opened in the middle of the first bottom surface 51, a second accommodation space 55 hollowed inward from the second opening 53, a conductive portion 56 of a porous structure formed on the top side of the second accommodation space 55 to provide a capillary action to connect the inner wall of the second accommodation space 55 to the entire porous diffuser 5, and a mounting portion 57 located om the conductive portion 56. The porous diffuser 5 utilizes the first bottom surface 51 to detachably combine with the connecting surface 31 of the accommodation base 3.

The liquid-conducting member 7 is made of porous material. The liquid-conducting member 7 has one thereof coupled to the mounting portion 57, and an opposite end thereof, namely, the free end 71 passing through the first opening 33 and the second opening 53 while protruding into the accommodation space 33.

In some embodiments, the material of the porous diffuser 5 includes porous ceramics, wood, paper, gypsum, vermiculite, rock, volcanic rock, diatomaceous earth, laterite, stone powder, foamed stone, sponge, non-woven fabric, fiber fabric or any kind of polymers.

The porous diffuser 5 is, for example, a porous ceramic structure formed by firing clay between 700° C. and 1100° C. It has excellent adsorption and release effects on liquids, can reduce weight, and at the same time, the overall structure can be greatly simplified, which is lightweight and has excellent fragrance liquid carrying and evaporation effects.

The liquid-conducting member 7 of the multi-pore aroma diffuser device 1 is made of cotton swabs, cotton cores, porous ceramics, wood, paper, sponges, non-woven fabrics, PET, fiber fabrics or polymers, and has good liquid adsorption and capillary transmission effects.

In some embodiments, the liquid is, for example, a functional liquid, including but not limited to any one of essential oils, essences, perfumes, deodorants, mosquito repellents, etc. The liquid-conducting member 7 is used to adsorb and conduct liquid to the conductive portion 56. The liquid can be transported to the inner wall of the conductive portion 56, the second accommodation space 55, and the entire porous diffuser 5 by utilizing the capillary action of the porous structure to release the essential oil fragrance into the space.

In a preferred embodiment, the liquid-conducting member 7 of a cotton swab (or cotton core) can be used to absorb liquid, such as functional liquid such as essential oil or perfume.

The porous cotton swab has good essential oil adsorption and capillary action, which can transport the essential oil to the inner wall of the second accommodation space 55 and the entire porous diffuser 5. By continuously providing fragrance liquid through the structure of porous materials such as the porous diffuser 5 made of the above-mentioned porous ceramics and the liquid storage bottle, referenced by 9, the fragrance is continuously emitted from the multi-pore aroma diffuser device 1 to improve the problem of aroma diffusion stones. There is no need to rely on power-consuming or energy-consuming heating incense machines to produce fragrance. It has a long service life, is energy-saving and environmentally friendly. At the same time, through the porous structure, such as porous diffuser 5, it absorbs various liquids, such as functional liquids, such as essential oils or perfumes or other liquid fragrances, etc., and diffuses fragrances slowly or at a controllable diffusion rate.

Please refer to FIGS. 1-4 again. A multi-pore aroma diffuser device 1 according to an embodiment of the present invention, wherein the shape of the porous diffuser 5 is a conical structure tapering from the side of the first bottom surface 51 toward the side of the first top surface 50. The liquid-conducting member 7 can controllably absorb the amount of liquid such as essential oil, and transport the essential oil at a capillary rate to the conductive portion 56, thereby effectively controlling the amount of essential oil used. At the same time, the conical structure of the porous diffuser 5 has a larger diameter on the side of the first bottom surface 51 and the diameter gradually decreases relatively towards the side of the first top surface 50. Therefore, when controlling the use of a smaller amount of essential oil, the capillary action of the conductive portion 56 is used to transport the essential oil to the direction of the first top surface 50 on the distal side, thereby effectively transmitting a small amount of essential oil from the conductive portion 56 to the distal first top surface 50, allowing a small amount of essential oil to be continuously distributed throughout the porous diffuser 5.

Correspondingly, the axial surface area of the porous diffuser 5 is a conical surface structure with the diameter gradually decreased from the side of the first bottom surface 51 towards the side of the first top surface 50, so that a small amount of essential oil can be continuously transported from the larger surface area side to the smaller surface side by capillary action, so as to continuously and fully supply the essential oil and distribute it throughout the axial surface area of the porous diffuser 5, so as to continuously evaporate the fragrance from the pores of the porous diffuser 5 and diffuse it into the space. With a lightweight device and excellent fragrance liquid carrying and evaporation effects, the diffusion of fragrance can be slow or at a controlled diffusion rate.

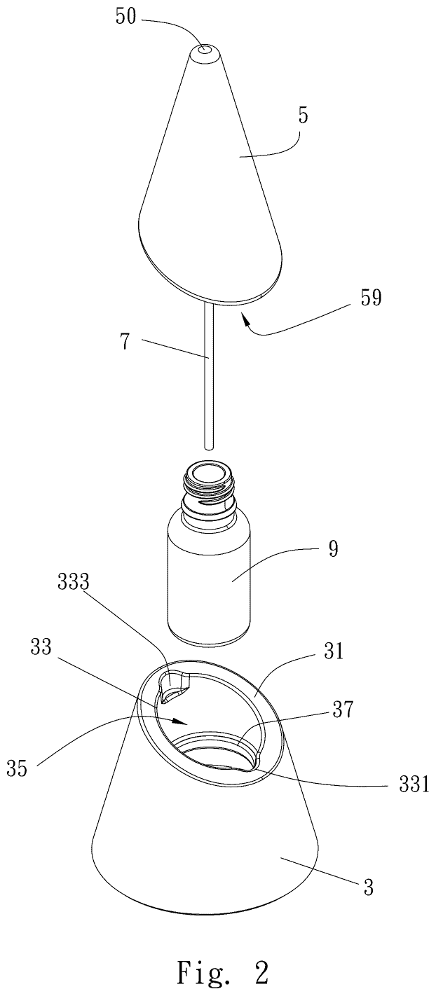

Please refer to FIGS. 2-4. The conical structure of the porous diffuser 5 of the multi-pore aroma diffuser device 1 according to some embodiments of the present includes a tapered conical structure. The first bottom surface 51 and second opening 53 of the porous diffuser 5 of the tapered conical structure are circular structures. The corresponding shapes of the connecting surface 31 and the first opening 33 of the accommodation base 3 match the shapes of the first bottom surface 51 and the second opening 53 of the porous diffuser 5, corresponding to a circular structure.

The conical structure shape of the porous diffuser 5 of the multi-pore aroma diffuser device according to some embodiments of the present invention includes an elliptical cone structure. The first bottom surface 51 and second opening 53 of the porous diffuser 5 of the elliptical cone structure are elliptical structures. The corresponding shapes of the connecting surface 31 and the first opening 33 of the accommodation base 3 match the shapes of the first bottom surface 51 and the second opening 53 of the porous diffuser 5, corresponding to an elliptical structure.

The conical structure shape of the porous diffuser 5 of the multi-pore aroma diffuser device according to some embodiments of the present invention includes a pyramidal structure. The first bottom surface 51 and second opening 53 of the porous diffuser 5 of the pyramidal structure are polygonal structures. The corresponding shapes of the connecting surface 31 and the first opening 33 of the accommodation base 3 match the shapes of the first bottom surface 51 and the second opening 53 of the porous diffuser 5, corresponding to a polygonal structure while the porous diffuser 5 thins the walls.

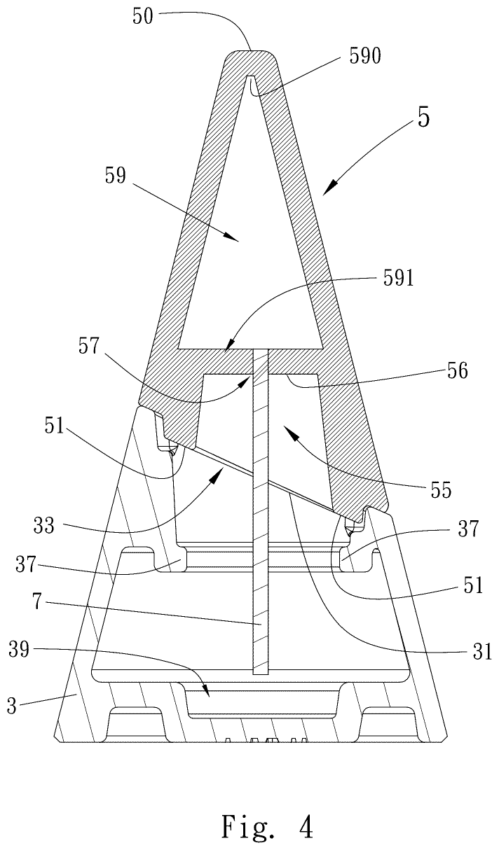

Please refer to FIG. 4. In the multi-pore aroma diffuser device 1 according to an embodiment of the present invention, the porous diffuser 5 further comprises a hollow portion 59, which is opened between the upper side of the conductive portion 56 and the first top surface 50. The hollow portion 59 forms a ventilated space while the porous diffuser 5 thins the walls. Therefore, the hollow portion 59 inside the porous diffuser 5 and the outside air are easily connected through the pores of the porous diffuser 5, and the air pressure inside the porous diffuser 5 can be balanced, so that. This further improves the capillary flow of liquids such as essential oils to the entire porous diffuser 5 and its surface pores, allowing the essential oil molecules to diffuse smoothly and more efficiently.

Please refer to FIGS. 2-4. In the multi-pore aroma diffuser device 1 according to an embodiment of the present invention, the hollow portion 59 of the porous diffuser 5 has a second bottom surface 591 and a second top surface 590 opposite to the second bottom surface 591. The second bottom surface 591 is defined from the upper side of the conductive portion 56. The shape of the hollow portion 59 is a structure that tapers from the second bottom surface 591 toward the second top surface 590. The surface area of the second bottom surface 591 on the bottom side of the hollow portion 59 is larger than the surface area of the second top surface 590. The multi-pore aroma diffuser device 1 of the present invention does not use electricity or heat energy to diffuse aroma, but only uses the capillary action of the liquid-conducting member 7 of cotton swab material to transmit essential oils to the pores of the porous diffuser 5 of porous ceramics. Capillary action is used to transmit essential oils and porous evaporation essential oil aromas. Therefore, the conductive portion 56 on the bottom side of the second bottom surface 591 of the hollow portion 59 can effectively capillaryly transfer a controlled amount of essential oil from the porous diffuser 5 with larger area and volume on the side of the second bottom surface 591 to the porous diffuser 5 with smaller area and volume on the side of the second top surface 590.

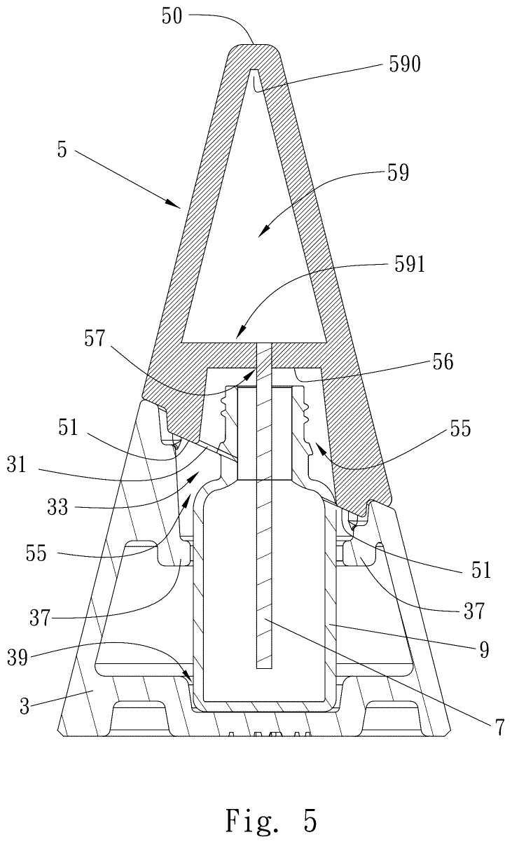

Please refer to FIGS. 4-5. In the multi-pore aroma diffuser device 1 according to an embodiment of the present invention, the hollow portion 59 of the porous diffuser 5 is a cone-shaped space-defined structure, and the bottom side of the cone-shaped hollow portion 59 is tapered toward the top, and its shape is like a triangular space. This triangular space structure is used to match the conical or elliptical conical or pyramidal porous diffuser 5, so that the wall thickness of the porous diffuser 5 outside the hollow portion 59 is even, and pore capillary action can be effectively carried out on the controlled amount of essential oil and the fragrance can be more smoothly evaporated.

Please refer to FIGS. 2-5. In the multi-pore aroma diffuser device 1 according to an embodiment of the present invention, the first bottom surface 51 further comprises a convex wall 52, which is formed by protruding from the area between the inner side of the first bottom surface 51 and the second opening 53. The convex wall 52 can be a structure of an annular convex wall or a plurality of spaced convex walls formed around the periphery of the second opening 53. The convex wall 52 is detachably engaged in the first opening 33 of the accommodation base 3. The convex wall 52 allows the user to detachably and quickly combine the porous diffuser 5 with the accommodation base 3. The porous diffuser 5 uses the convex wall 52 to be mounted on the first opening 33 and is not easily separated from the accommodation base 3, thereby achieving a detachable design of the multi-pore aroma diffuser device 1. And it is convenient to put a liquid storage bottle 9 into the accommodation base 3 or to replace the liquid storage bottle 9 for use, thereby improving the dilemma of traditional aroma diffuser stones needing to replenish essential oils repeatedly.

Please refer to FIGS. 2-5. In the multi-pore aroma diffuser device 1 according to an embodiment of the present invention, a first rib 521 extends outward from one end edge of the convex wall 52, and a first fixing groove 331 is recessed inside the first opening 33 of the accommodation base 3. The first rib 521 is used to be detachably combined in the first fixing groove 331 for fixation and fool-proofing. When the user combines the porous diffuser 5 with the accommodation base 3, the first rib 521 can detachably engage with the first fixing groove 331 to prevent fooling, and at the same time, the porous diffuser 5 will not slide relative to the accommodation base 3.

Please refer to FIGS. 2-5. In the multi-pore aroma diffuser device 1 according to an embodiment of the present invention, a second rib 523 extends outward from the convex wall 52 relative to the outer edge of one end of the first rib 521, and the first opening 33 of the accommodation base 3 and the first fixing groove 331 are concave with a second fixing groove 333 on the opposite side. The second rib 523 is used to be detachably combined in the second fixing groove 331 for fixation and fool-proofing. Just like the function of the first rib 521 mentioned above, the second rib 523 further detachably engages with the second fixing groove 333 to prevent fooling. At the same time, the porous diffuser 5 will not slide relative to the accommodation base 3, and the combination of the two is more stable.

Please refer to FIGS. 1-5. In the multi-pore aroma diffuser device 1 according to an embodiment of the present invention, the connecting surface 31 and the first opening 33 are inclined from one side to the other, whereby the upper side of the accommodation base 3 is formed into an inclined structure. Correspondingly, the first bottom surface 51 and the second opening 53 are inclined from one side to the other side. Thereby, the lower side of the porous diffuser 5 is formed into a slope structure, which is used to detachably combine with the slope structure on the upper side of the accommodation base 3. The aforementioned convex wall 52 is detachably clamped in the first opening 33, the first rib 521 is clamped in the first fixing groove 331, and the second rib 523 is clamped in the second fixing groove 333, so that the sloped structure of the porous diffuser 5 can be quickly and conveniently installed on the upper sloped structure of the accommodation base 3 and will not slide. The combination of the two can prevent fooling as mentioned above, and can achieve a detachable design, which is convenient for replacing a liquid storage bottle 7 inside the multi-pore aroma diffuser device 1. As shown in FIG. 1, the appearance of the multi-pore aroma diffuser device 1 is as beautiful as a simple Christmas tree, which is also different from the traditional diffuser stone structure shape.

Please refer to FIGS. 1-3. In the multi-pore aroma diffuser device 1 according to an embodiment of the present invention, the conical structure of the porous diffuser 5 includes an elliptical cone structure, and the first bottom surface 51, second opening 53 and the convex wall 52 of the elliptical cone structure of the porous diffuser 5 are elliptical structures. The corresponding shape of the connecting surface 31 and the first opening 33 of the accommodation base 3 matches the shape of the first bottom surface 51 and the second opening 53 of the porous diffuser 5, corresponding to the elliptical structure.

Please refer to FIGS. 2-4. In an embodiment of the present invention, the multi-pore aroma diffuser device 1 further comprises a liquid storage bottle 9. The liquid storage bottle 9 is used to be detachably combined in the first accommodation space 35 and the second accommodation space 55. The free end side 71 of the liquid-conducting member 7 penetrates deeply into the liquid (such as essential oil, perfume, etc.) in the liquid storage bottle 9 to attract the essential oil liquid and transport the essential oil to the conductive portion 56. Through the porous capillary action of the conductive portion 56, the liquid is transported and guided to the entire porous diffuser 5 to distribute fragrance in the space.

Please refer to FIGS. 2-5. In the multi-pore aroma diffuser device 1 according to an embodiment of the present invention, the porous diffuser 5 further comprises a sealing layer 59 coated on the first bottom surface 51. The sealing layer 59 is used to prevent the liquid of the porous diffuser 5 from leaking downward. The anti-seepage coating of the sealing layer 59 includes but is not limited to any anti-seepage material such as glaze, waterproof glue, adhesive, plastic, rubber or paint.

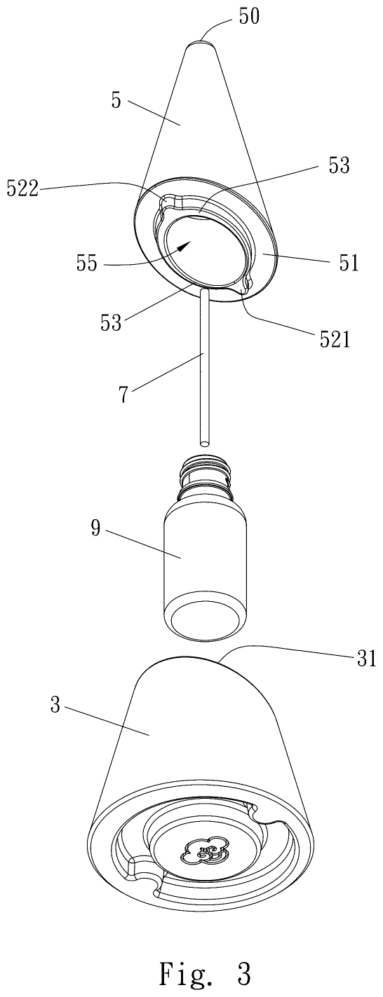

Please refer to FIGS. 3-5. In the multi-pore aroma diffuser device 1 according to an embodiment of the present invention, the accommodation base 3 further comprises a clamping portion 37 and an accommodating groove 39. The clamping portion 37 protrudes from the inside of the first accommodation space 35, and the accommodating groove 39 is recessed on the bottom side of the first accommodation space 35. The multi-pore aroma diffuser device 1 further comprises a liquid storage bottle 9, which is detachably combined in the first accommodation space 35 and the second accommodation space 55. The bottom of the liquid storage bottle 9 is detachably coupled to the accommodating groove 39. The clamping portion 37 detachably clamps the outside of the liquid storage bottle 9. The free end side 71 of the liquid-conducting member 7 is deep into the liquid in the liquid storage bottle 9 to attract the liquid and transport the liquid to the conductive portion 56. The liquid is transported and guided to the entire porous diffuser 5 through the porous capillary action of the conductive portion 56 to distribute fragrance in the space.

In summary, the multi-pore aroma diffuser device in some embodiments of the present invention continuously provides aroma liquid through the ceramic porous diffuser and the liquid storage bottle. It is light, simple in structure, easy to use and has excellent fragrance evaporation effect. It can effectively absorb the retained essential oil and then release it. There is no need to go through complicated steps to add essential oil, which greatly improves the convenience of use.

What the invention claimed is:

1. A multi-pore aroma diffuser device, comprising:
an accommodation base comprising a connecting surface formed on an upper side thereof and a first opening opened in a middle of said connecting surface, said first opening extended inward to form a first accommodation space;
a porous diffuser comprising a first top surface, a first bottom surface relative to said first top surface, a second opening formed in a middle of said first bottom surface, a second accommodation space hollowed inward from said second opening, a conductive portion formed in a top side within said second accommodation space and a mounting portion located on said conductive portion, said porous diffuser utilizing said first bottom surface to detachably combine with said connecting surface of said accommodation base; and
a liquid-conducting member, said liquid-conducting member being a porous material, said liquid-conducting member having one side thereof combined with said mounting portion and an opposing free end side thereof passed through said first opening and said second opening and protruded into said accommodating space.

2. The multi-pore aroma diffuser device as claimed in claim 1, wherein the material of said porous diffuser is selected from the group of porous ceramics, wood, paper, gypsum, vermiculite, rock, volcanic rock, diatomaceous earth, laterite, stone powder, foamed stone, sponge, non-woven fabric, fiber fabric and polymers.

3. The multi-pore aroma diffuser device as claimed in claim 1, wherein said porous diffuser is a porous ceramic structure formed by firing clay between 700° C.˜1100° C.

4. The multi-pore aroma diffuser device as claimed in claim 1, wherein the material of said liquid-conducting member is selected from the group of cotton swabs, cotton cores, porous ceramics, wood, paper, sponges, non-woven fabrics, PET, fiber fabrics and polymers.

5. The multi-pore aroma diffuser device as claimed in claim 1, wherein the shape of said porous diffuser is a conical structure that tapers from said first bottom surface toward said first top surface.

6. The multi-pore aroma diffuser device as claimed in claim 1, wherein said porous diffuser is a tapered surface structure in which the axial surface area tapers from said first bottom surface toward said first top surface.

7. The multi-pore aroma diffuser device as claimed in claim 5, wherein said conical structure of said porous diffuser is a tapered conical structure; said first bottom surface and said second opening of said porous diffuser of said tapered conical structure are circular structures; the shapes of said connecting surface and said first opening of said accommodation base match the shapes of said first bottom surface and said second opening shape of said porous diffuser, corresponding to a circular structure.

8. The multi-pore aroma diffuser device as claimed in claim 5, wherein said conical structure of said porous diffuser is an elliptical cone structure; said first bottom surface and said second opening of said porous diffuser of said elliptical cone structure are elliptical structures; the shapes of said connecting surface and said first opening of said accommodation base match the shapes of said first bottom surface and said second opening shape of said porous diffuser, corresponding to an elliptical structure; or wherein said conical structure of said porous diffuser is a pyramidal structure; said first bottom surface and said second opening of said porous diffuser of said pyramidal structure are polygonal structures; the shapes of said connecting surface and said first opening of said accommodation base match the shapes of said first bottom surface and said second opening shape of said porous diffuser, corresponding to a polygonal structure.

9. The multi-pore aroma diffuser device as claimed in claim 5, wherein said porous diffuser further comprises a hollow portion opened between an upper side of said conductive portion and said first top surface.

10. The multi-pore aroma diffuser device as claimed in claim 9, wherein said hollow portion of said porous diffuser comprises a second bottom surface and a second top surface relative to said second bottom surface, said second bottom surface being defined from an upper side of said conductive portion, the shape of said hollow portion being a structure that tapers from said second bottom surface toward said second top surface, the surface area of said second bottom surface on a bottom side of said hollow portion being larger than the surface area of said second top surface.

11. The multi-pore aroma diffuser device as claimed in claim 9, wherein said hollow portion of said porous diffuser is a cone-shaped space-defined structure.

12. The multi-pore aroma diffuser device as claimed in claim 1, wherein said porous diffuser further comprises a hollow portion opened between an upper side of said conductive portion and said first top surface, said hollow portion of said porous diffuser comprising a second bottom surface and a second top surface relative to said second bottom surface, said second bottom surface being defined from an upper side of said conductive portion.

13. The multi-pore aroma diffuser device as claimed in claim 5, wherein said first bottom surface further comprises a convex wall formed by protruding from the area between an inner side of said first bottom surface and said second opening, said convex wall being selectively a structure of an annular convex wall or a plurality of spaced convex walls formed around the periphery of said second opening, said convex wall being detachably engaged in said first opening of said accommodation base.

14. The multi-pore aroma diffuser device as claimed in claim 13, wherein said accommodation base further comprises a first fixing groove recessed on the inside of said first opening; said porous diffuser accommodation base further comprises a first rib extended outward from one end edge of said convex wall and detachably combined in said first fixing groove for fixation and fool-proofing.

15. The multi-pore aroma diffuser device as claimed in claim 14, wherein said accommodation base further comprises a second fixing groove recessed within said first opening a second fixing groove on the opposite side to said first fixing groove; said accommodation base further comprises a second rib extended outward from said convex wall relative to an outer edge of one end of said first rib. and detachably combined in said second fixing groove for fixation and fool-proofing.

16. The multi-pore aroma diffuser device as claimed in claim 15, wherein said connecting surface and said first opening are inclined from one side thereof to an opposite side thereof, so that said accommodation base has an upper side thereof forming an inclined structure; correspondingly, said first bottom surface and said second opening are inclined from one side thereof to an opposite side thereof, so that said porous diffuser has a lower side thereof formed into a sloped structure for detachable combination with the inclined structure on the upper side of said accommodation base.

17. The multi-pore aroma diffuser device as claimed in claim 16, wherein said conical structure of said porous diffuser is an elliptical cone structure, and said first bottom surface, said second opening and said convex wall of said elliptical cone structure of said porous diffuser are elliptical structures; the shapes of said connecting surface and said first opening of said accommodation base match the shapes said first bottom surface and said second opening of said porous diffuser, corresponding to an elliptical structure.

18. The multi-pore aroma diffuser device as claimed in claim 17, further comprising a liquid storage bottle detachably combined in said first accommodation space and said second accommodation space and holding a liquid therein, wherein said liquid-conducting member has a free end side thereof goes deep into said liquid in said liquid storage bottle to attract said liquid and transport said liquid to said conductive portion, so that said liquid is transported and guided to the entire said porous diffuser through a porous capillary action of said conductive portion to distribute fragrance in the space.

19. The multi-pore aroma diffuser device as claimed in claim 1, wherein said porous diffuser further comprises a sealing layer coated on said first bottom surface, said sealing layer being formed of an anti-seepage material selected from the group of glaze, waterproof glue, adhesive, plastic, rubber and paint.

20. The multi-pore aroma diffuser device as claimed in claim 1, further comprising a liquid storage bottle detachably combined in said first accommodation space and said second accommodation space and holding a liquid therein, wherein said accommodation base further comprises a clamping portion and an accommodating groove, said clamping portion protruded from the inside of said first accommodation space to detachably clamp said liquid storage bottle, said accommodating groove being recessed on a bottom side of said first accommodation space to detachably hold a bottom side of said liquid storage bottle; said liquid-conducting member has a free end side thereof goes deep into said liquid in said liquid storage bottle to attract said liquid and transport said liquid to said conductive portion, so that the liquid is transported and guided to the entire said porous diffuser through a porous capillary action of said conductive portion to distribute fragrance in the space.
